(19) United States
(12) Patent Application Publication
(10) Pub. No.: US 2014/0303498 A1
(43) Pub. Date: Oct. 9, 2014

(54) OBJECT INFORMATION ACQUISITION APPARATUS, OBJECT INFORMATION ACQUISITION SYSTEM, DISPLAY CONTROL METHOD, DISPLAY METHOD, AND PROGRAM

(71) Applicants: Tadaki Watanabe, Tokyo (JP); Koichiro Wanda, Yokohama-shi (JP)

(72) Inventors: Tadaki Watanabe, Tokyo (JP); Koichiro Wanda, Yokohama-shi (JP)

(73) Assignee: CANON KABUSHIKI KAISHA, Tokyo (JP)

(21) Appl. No.: 14/237,339

(22) PCT Filed: Jul. 30, 2012
(86) PCT No.: PCT/JP2012/004831

§ 371 Date: May 27, 2014

(30) Foreign Application Priority Data
Aug. 8, 2011 (JP) 2011-172974
Jul. 12, 2012 (JP) 2012-156629

(51) Int. Cl. A61B 5/00

(52) U.S. Cl. CPC A61B 5/0095; A61B 5/708; A61B 5/6843
USPC 600/437

(57) ABSTRACT

In a known apparatus, when a user defines a specified region in which characteristic information is to be acquired, it may be difficult for the user to easily recognize a corresponding scanning region that is to be actually scanned with a receiver. An embodiment of the invention provides an object information acquisition apparatus including a receiver configured to receive an acoustic wave and convert it into an electric signal, a scan control unit configured to scan the receiver at least in one direction, and a display control unit. Upon receiving information associated with a region specified to be scanned to acquire characteristic information, the display control unit outputs information for displaying an adjusted region obtained by adjusting the specified region such that a length of the specified region in a first direction corresponds to an integral multiple of a scanning width of the receiver in the first direction.

### TECHNICAL FIELD

The present invention relates to an object information acquisition apparatus for use in medical diagnosis, nondestructive inspection, etc., an object information acquisition system, a display control method, a display method, and a program.

### BACKGROUND ART

A photo acoustic imaging (PAI) technique is known as one of photo imaging techniques using light. In the PAI technique, a living body given as an object is illuminated with light, and an acoustic wave, which is generated when photo energy is absorbed by a part to be examined such as a tumor, is received by a receiver. A reception signal output from the receiver is analyzed and optical characteristic information of the inside of the living body is acquired as image data.

PTL 1 discloses an apparatus configured to hold a breast from both sides thereof with a holding unit and receive an acoustic wave while two-dimensionally scanning a receiver over the holding unit. By two-dimensionally scanning the receiver, it is possible to obtain characteristic information at a plurality of positions in the object.

### CITATION LIST

### Patent Literature

PTL 1: Japanese Patent Laid-Open No. 2010-022812

### SUMMARY OF INVENTION

In a case where the receiver is two-dimensionally scanned in a main scanning direction and a sub scanning direction as with the technique disclosed in PTL 1, scanning may be performed not over a whole scannable region but only within a particular region specified by a user or an operator to acquire characteristic information of this region in the object. More specifically, an image of the object in a state in which it is held by the holding unit may be taken by a camera, and the image thereof may be displayed on a display screen such that a user or an operator may specify a particular region while referring to the image displayed on the display screen.

However, a reception time period needed to receive an acoustic wave for acquiring characteristic information of the specified region (the time period necessary to receive the acoustic wave at all scanning positions) is not necessarily proportional to the size of the specified region. In particular, when the receiver is scanned at a low speed, the reception time period varies greatly depending on the number of times the main scanning and the sub scanning are performed to acquire characteristic information of the specified region. Typically, the number of scans increases or decreases in a stepwise manner with an increase or reduction in the specified region, and the reception time period correspondingly increases or decreases. However, in known techniques, when a specified region is given, it is difficult for a user to recognize a corresponding scanning region that is to be actually scanned with the receiver.

Therefore, a slight difference in the specified region may result in an unnecessary increase in the number of main or sub scans of the receiver, which may create a redundant time period in the acoustic wave reception time period for acquiring characteristic information, which may in turn redundantly increase a time period during which a person under examination is constrained.

In view of the above, the present invention provides a technique that allows a user to recognize an actually necessary scanning region of the receiver corresponding to the specified region defined by the user.

In an aspect, the present invention provides an object information acquisition apparatus, configured to receive an acoustic wave from an object and acquire characteristic information of the object, including a receiver configured to receive the acoustic wave and convert the received acoustic wave into an electric signal, a scan control unit configured to scan the receiver at least in one direction, and a display control unit, wherein the display control unit receives information associated with a specified region defined by a user as a region in which characteristic information is to be acquired, and the display control unit outputs information for displaying an adjusted region obtained by adjusting the specified region such that a length of the specified region in a first direction corresponds to an integral multiple of a scanning width of the receiver in the first direction.

In an aspect, the invention provides a method of controlling displaying a region in which characteristic information of an object is to be acquired, including receiving information associated with a specified region defined by a user as the region in which the characteristic information of the object is to be acquired, and outputting information for displaying, on a display unit, an adjusted region obtained by adjusting the specified region such that at least a length of the specified region in a first direction corresponds to an integral multiple of a scanning width, in the first direction, of a receiver configured to receive an acoustic wave from the object. In an aspect, the invention provides a method of displaying a region in which characteristic information of an object is to be acquired, including displaying a captured image of the object, and displaying a specified region and an adjusted region, the specified region defined by a user as the region in which the characteristic information of the object is to be acquired, the adjusted region obtained by adjusting the specified region such that at least a length of the specified region in a first direction corresponds to an integral multiple of a scanning width, in the first direction, of a receiver configured to receive an acoustic wave from the object.

According to the aspects of the invention, in operations of scanning the receiver and receiving acoustic waves, a user is allowed to easily recognize an actually necessary scanning region of the receiver corresponding to a specified region defined by the user.

### BRIEF DESCRIPTION OF DRAWINGS

FIG. 1 is a block diagram of an object information acquisition apparatus according to an embodiment of the present invention.

FIG. 2A is a conceptual diagram illustrating an example of a method of defining a specified region according to an embodiment of the present invention.

FIG. 2B is a conceptual diagram illustrating an example of a three-dimensional region corresponding to a specified region.

FIG. 3 is a flow chart illustrating an example of a display control method according to an embodiment of the present invention.

FIG. 4 is a conceptual diagram illustrating a specified region in a scanning coordinate system according to a first embodiment.

FIG. 5 is a conceptual diagram illustrating a specified region and an adjusted region according to the first embodiment.

FIG. 6 is a conceptual diagram illustrating a specified region in a scanning coordinate system according to a second modified example of the first embodiment.

FIG. 7 is a conceptual diagram illustrating a specified region in a scanning coordinate system according to a second embodiment.

FIG. 8 is a conceptual diagram illustrating a specified region and an adjusted region according to the second embodiment.

### DESCRIPTION OF EMBODIMENTS

A basic idea of the present invention is based on a fact that a scanning region of a receiver necessary to cover a region specified by a user is determined by a scanning width of the receiver. As will be described in detail below with reference to embodiments, when a region (specified region) is specified by a user as a region in which characteristic information is to be acquired, an adjustment is performed such that at least a length of the specified region in one direction corresponds to an integral multiple of the scanning width of the receiver, and a resultant region is displayed as an adjusted region on a display unit.

In the embodiments of the present invention, the acoustic wave may be an elastic wave such as a sonic wave, an ultrasonic wave, a photoacoustic wave, a photo-ultrasonic wave, or the like, and the receiver receives the acoustic wave propagating through the object. That is, in the embodiments of the present invention, the object information acquisition apparatus includes an apparatus using a photoacoustic effect configured to illuminate an object with light (an electromagnetic wave) thereby generating an acoustic wave in the object, and receive the generated acoustic wave and acquire characteristic information in the object. In the case of the apparatus using the photoacoustic effect, examples of acquired characteristic information in the object include an initial sound pressure of an acoustic wave generated by illumination of light, an absorption density or an absorption coefficient of photo energy determined from the initial sound pressure, object information depending on a content or the like of a substance forming a tissue, etc. Examples of substance contents include an oxygen saturation, oxy/deoxyhemoglobin content, etc. The characteristic information may be acquired in the form of numerical data, or the characteristic information may be acquired in the form of a characteristic distribution at various positions in the object, i.e., in the form of image data indicating a distribution in terms of an absorption coefficient, an oxygen saturation, etc.

In the embodiments of the invention, the object information acquisition apparatus may be implemented as an apparatus using an ultrasonic wave echo technique configured to transmit an ultrasonic wave to an object and receive a reflected wave from the inside of the object thereby acquiring characteristic information of the inside of the object. In the case of the apparatus using the ultrasonic wave echo technique, the characteristic information to be acquired is information in which a difference in acoustic impedance of a tissue inside the object is reflected.

Specific embodiments of the invention are described below with reference to drawings. Note that similar constituent parts are denoted by similar reference numerals or symbols, and duplicated explanations are not given.

### First Embodiment

In a first embodiment described below, an apparatus using a photoacoustic effect is disclosed. The apparatus includes a receiver scanned in a main scanning direction and a sub scanning direction. When a region (specified region) is specified by a user, a length of the specified region in the sub scanning direction and a resultant region is displayed as an adjusted region. In the following description, a basic configuration of the apparatus and functions thereof are first explained. Thereafter, a method of defining the specified region and a display control method including features of the embodiment of the invention are explained.

### Basic Configuration of Apparatus

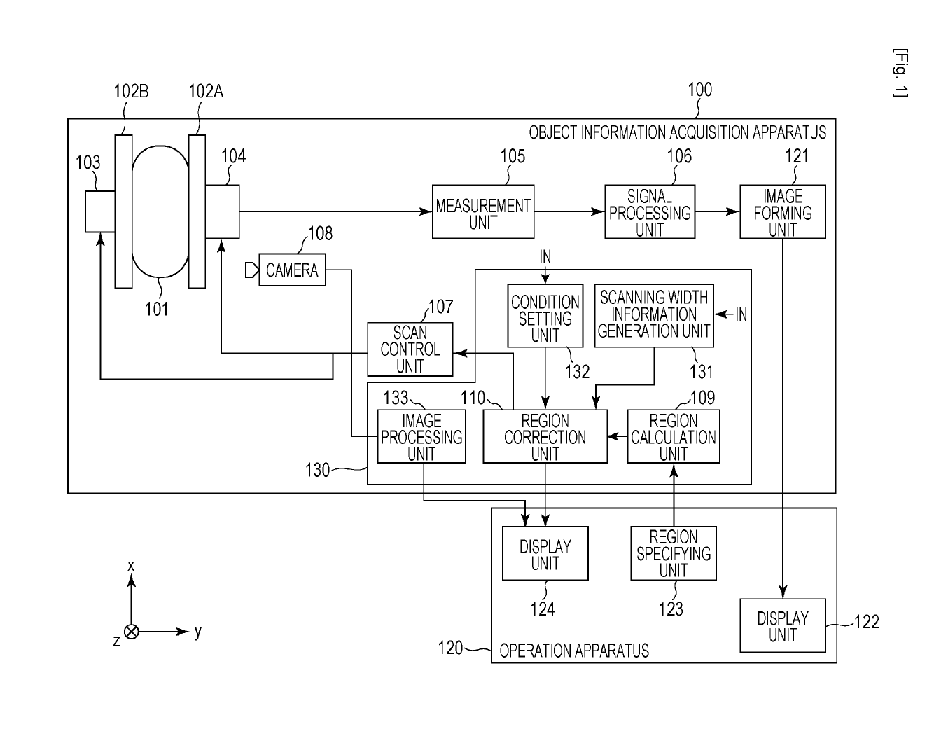

FIG. 1 is a schematic diagram illustrating a configuration of an object information acquisition system including an object information acquisition apparatus 100 and an external operation apparatus 120 according to the first embodiment.

The object information acquisition apparatus 100 according to the present embodiment includes a holding unit 102 configured to hold a living body 101 given as an object, an illumination unit 103 configured to emit illumination light, a receiver 104 configured to receive an acoustic wave and convert it into a reception signal, and a measurement unit 105 configured to amplify the reception signal and convert it into a digital signal. The object information acquisition apparatus 100 further includes a signal processing unit 106 configured to perform a cumulative addition process or the like on the reception signal converted into the digital signal, an image forming unit 121 configured to generate image data using an output signal provided from the signal processing unit 106, a display control unit 130, a scan control unit 107 configured to control the scanning of the receiver 104, and a camera 108 serving as an image capturing unit.

The operation apparatus 120 includes a display unit 124 configured to display a captured image, a region specifying unit 123 for use by a user to define a specified region, and a display unit 122 configured to display an image generated by the image forming unit 121.

Elements of the object information acquisition apparatus 100 are described in further detail below.

### Holding Unit

The holding unit 102 includes a pair of a first holding unit 102A and a second holding unit 102B between which a living body 101 such as a breast is put and held from both sides of the living body 101. The relative positions of these two holding units 102A and 102B are controlled by a holding mechanism (not shown) to adjust a holding distance and a holding pressure. In the following description, the holding units 102A and 102B are generically denoted as the holding unit 102 unless it is necessary to distinguish between them.

By firmly holding the living body 101 with the holding unit 102, it is possible to reduce a measurement error caused by motion of the living body 101. Furthermore, use of the holding unit 102 makes it possible to properly adjust the thickness of the living body 101 depending on a penetration depth of light. The holding unit 102 is located in the middle of an optical path, and thus the holding unit 102 may be formed using a material such as polymethylpentene or the like highly transparent to light used. The holding unit 102A located closer to the receiver 104 may be formed of a material that provides good acoustic matching with the receiver 104.

### Illumination Unit

The illumination unit 103 that illuminates the living body 101 with light includes a light source configured to emit light, and an illumination part configured to guide the light emitted from the light source to the object such that the object is illuminated with the light. As for the light source, a solid-state laser may be used which is capable of emitting pulsed light (with a pulse width equal to or less than 100 nsec) having a center wavelength in a near-infrared range from 530 to 1300 nm. Examples of such solid-state lasers include a Yttrium-Aluminium-Garnet laser, a Titan-Sapphire laser, etc. The wavelength of light may be selected in a range from 530 nm to 1300 nm depending on a light absorbing substance (such as hemoglobin, glucose, cholesterol, etc.) in the living body to be examined Examples of the illumination part include an optical reflection mirror, a lens that focuses or expands light or changes a shape of light, a prism that disperses, refracts, or reflects light, an optical fiber that transmits light, a diffusion plate, etc. Any illumination part may be used as long as it is capable of illuminating a desired area of the object with light with a desired form emitted from the light source. The position of a light emerging end of the illumination part (i.e., illumination area) is controlled by the scan control unit 107.

### Receiver

The receiver 104 includes a plurality of elements for receiving an acoustic wave from the object and converting it into an electric signal (reception signal). Examples of devices usable as elements of the receiver 104 include a transducer using a piezoelectric effect, a transducer using optical resonance, a transducer using a change in electrostatic capacitance, etc. Any type of device may be used as elements as long as it is capable of receiving an acoustic wave and converting it into an electric signal. The sound pressure of the generated acoustic wave is proportional to the intensity of light. Therefore, the signal-to-noise ratio (SNR) of the reception signal may be increased by employing a configuration in which light illuminates a front region of the receiver. To this end, the illumination unit 103 and the receiver 104 may be located such that the light emerging end of the illumination unit 103 opposes the receiver 104 via the object. The scan control unit 107 may synchronously perform scanning such that the positional relationship between the light emerging end and the receiver 104 is maintained. The illumination part may guide light such that the living body 101 is also illuminated from the side of the receiver 104.

### Measurement Unit

The measurement unit 105 includes a signal amplifier that amplifies an analog signal (analog reception signal) input from the receiver 104, and an analog-to-digital converter that converts the analog signal into a digital signal. The signal amplifier controls a gain depending on a time since the object is illuminated with light till the acoustic wave reaches the elements of the receiver so that uniform contrast is obtained regardless of the depth in the object (living body).

### Signal Processing Unit

The signal processing unit 106 performs processes including correcting difference among elements in terms of sensitivity to the digital reception signal output from the measurement unit 105, making up for a lost signal due to a physical or electric defect of an element, storing the signal in a storage medium (not shown), cumulatively adding signals to reduce noise, etc. In the cumulative addition process, an acoustic wave from the living body 101 is received repeatedly at the same scanning position and the received signal is cumulatively added and averaged thereby reducing system noise. This makes it possible to obtain a reception signal with improved SNR.

### Image Forming Unit

The image forming unit 121 serves as a characteristic information acquisition unit that acquires image data representing a distribution of optical characteristic information (absorption coefficient, oxygen saturation, etc.) at various locations in the living body 101 using a signal output from the signal processing unit 106. The generated image data may be subjected, as required, to various correction processes such as luminance adjustment, distortion correction, clipping of a particular region of interest, etc., to obtain image data more suitable for diagnosis. Furthermore, to reduce noise, characteristic information acquired at the same location may be subjected to a cumulative combination process. The display unit 122 receives the image data from the image forming unit 121 and displays an image of the characteristic distribution based on the received image data.

### Scan Control Unit

As described above, the scan control unit 107 is a unit configured to control the light emerging end and the scanning position of the receiver 104. By performing two-dimensional scanning on the living body 101 and receiving acoustic waves at respective scanning positions, it is possible to obtain characteristic information over a wide range even when the receiver is of a small type. In the present embodiment, the scanning of the receiver is not limited to the two-dimensional scanning but the scanning may be performed in other manners as long as the receiver is scanned at least in one direction. The scan control unit 107 may change the scanning region depending on a result of controlling of the display control unit 130.

### Image Capturing Unit

The camera 108 serving as the image capturing unit is for capturing an image of the living body 101 and is installed such that its line-of-sight direction is perpendicular to the holding unit 102 for holding the living body 101. The captured image is transmitted to the display unit 124 via a captured image processing unit 133. The field of view of the camera 108 may be set such that the captured image covers the whole region scannable by the receiver 104. The captured image is displayed on the display unit 124 such that a user is allowed to refer to the captured image displayed on the display unit 124 during the operation of specifying a region (specified region) in which characteristic information is to be acquired. In the present embodiment, the display unit 124 is provided separately from the display unit 122 for displaying an image of characteristic information. However, alternatively, the image for use in defining the specified region and the image of characteristic information may be displayed on a single display unit.

### Region Specifying Unit

The region specifying unit 123 is an input unit for use by a user to define a specified region. The user is allowed to input a specified region while referring to a captured image of the living body 101 displayed on the display unit 124. Specific examples of the region specifying unit 123 include a pointing device such as a mouse, a keyboard, or the like, a tablet-type device, a touch pad attached to a surface of the display unit 124, etc.

### Display Control Unit

The display control unit 130 receives information associated with the specified region defined by a user and outputs information for displaying, on the display unit, an adjusted region obtained by adjusting the specified region such that at least a length in a first direction of the specified region corresponds to an integral multiple of a scanning width of the receiver. In the present embodiment, the display control unit 130 includes a region calculation unit 109 configured to convert the expression of the specified region from a display coordinate system into a scanning coordinate system, and a region correction unit 110 configured to correct at least the length in the first direction of the specified region based on at least the scanning width in the first direction of the receiver. The display control unit 130 also includes a captured image processing unit 133 configured to output image data captured by the camera to the display unit 124.

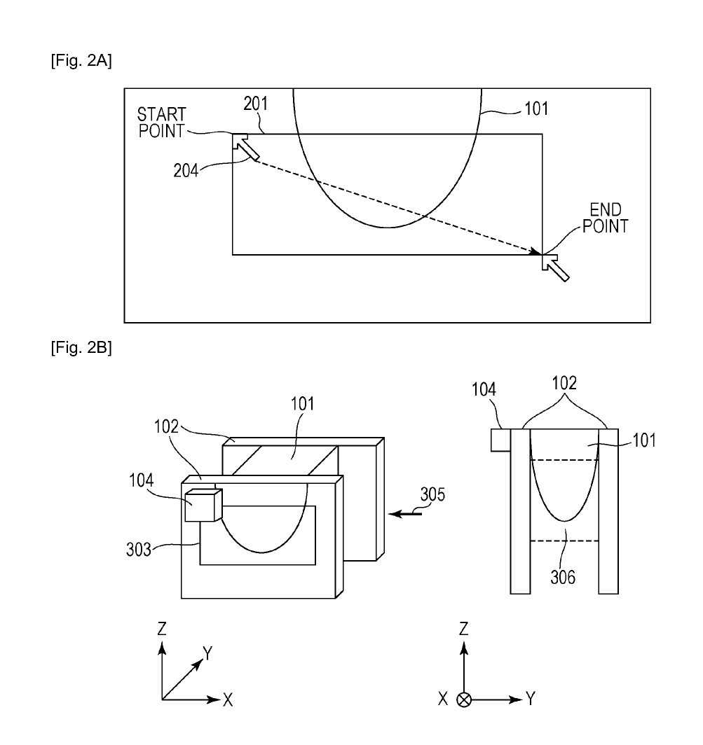

In the present embodiment, the region correction unit 110 corrects the length of the specified region in the sub scanning direction so as to be equal to the length of a scanning region (first scanning region) in the sub scanning direction that is to be scanned by the receiver to acquire the characteristic information of the specified region or the length of a second scanning region in the sub scanning direction in which the number of sub scans is smaller by one than in the first scanning region. More specifically, the correction is performed such that the length of the specified region in the sub scanning direction corresponds to an integral multiple of the sub-scanning width of the receiver. In the following description of the present embodiment, it is assumed, by way of example, that the first direction is the sub scanning direction and the second direction is the main scanning direction. The display control unit 130 is described in further detail below with reference to FIGS. 2A and 2B and FIGS. 3 to 5. Note that in the present embodiment, the main scanning direction is a direction in which the receiver is scanned while receiving an acoustic wave at each reception position, and the sub scanning direction is a direction perpendicular to the main scanning direction.

Note that in alternative embodiments of the present invention, the length of the specified region in the main scanning direction may be corrected (as in a second embodiment described later), or the length may be corrected in both the main scanning direction and the sub scanning direction (as in a third embodiment described later).

Furthermore, in alternative embodiments of the present invention, instead of producing the adjusted region by correcting the length of the specified region, an adjusted region may be newly produced based on information associated with the length of the specified region (such that, for example, the length of the adjusted region may be equal to an integral multiple of a scanning width). That is, the correction is not limited to that performed by the display control unit 131. It is sufficient for the display control unit 131 to be capable of producing information for displaying the adjusted region on the display unit. In a case where the specified region defined by a user is equal to an integral multiple of the scanning width of the receiver, the information associated with the specified region may be directly output as information associated with the adjusted region. Note that the “adjustment” is not limited to that which is performed so as to achieve exact coincidence of length. For example, an error may be allowed up to 2 to 3 times an element size of the receiver, or an error may be allowed as long as it is possible to correctly recognize the correspondence between the displayed region and the region actually scanned.

In the present embodiment, as shown in FIG. 1, the display control unit 130 may include a scanning-width information generation unit 131 configured to generate information associated with the scanning width of the receiver, and a condition setting unit 132 for setting a condition associated with a cumulative addition process on reception signals or a cumulative combination process on characteristic information (image data), etc.

The scanning-width information generation unit 131 generates information associated with the scanning width in accordance with a command issued by a user. In a case where the sub-scanning width and the main scanning width are fixed, it does not necessarily need to provide the scanning-width information generation unit 131. However, the sub-scanning width and the main scanning width may vary depending on the number of operations of cumulatively adding reception signals to improve the SNR or the number of operations of combining characteristic information. Therefore, in accordance with such setting for improving SNR, the scanning-width information generation unit 131 determines the scanning width and transmits information associated with the scanning width to the region correction unit 110, as will be described in further detail later with reference to a second modified example.

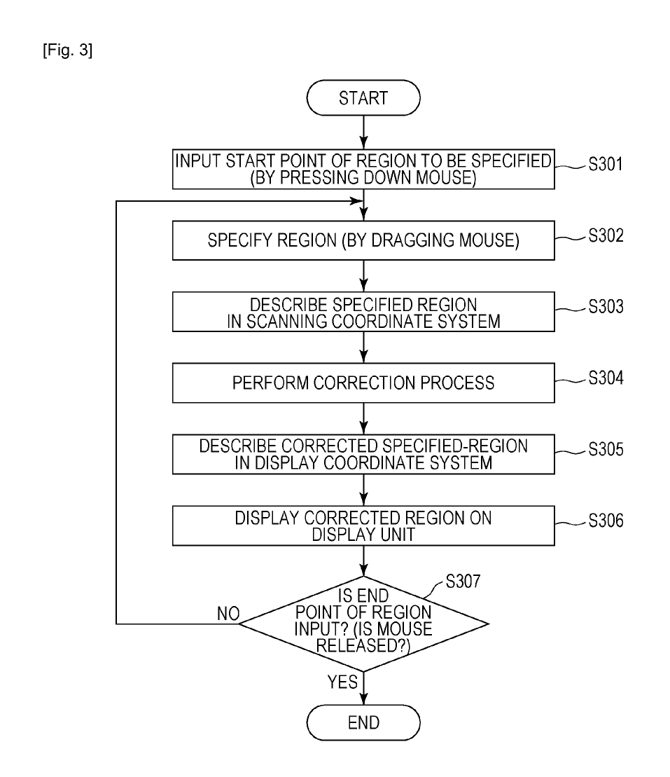

The condition setting unit 132 sets a condition in terms of the adjustment of the display control unit 130. More specifically, in the present embodiment, the condition setting unit 132 specifies whether the length of the specified region in the sub scanning direction is adjusted so as to be equal to the length of a scanning region (first scanning region) in the sub scanning direction that is to be scanned by the receiver to acquire the characteristic information of the specified region or the length of a second scanning region in the sub scanning direction in which the number of sub scans is smaller by one than in the first scanning region. The condition setting will be described in further detail later with reference to FIGS. 3 to 5.

The configuration of the object information acquisition system according to the present embodiment has been described above. Note that in the example shown in FIG. 1, the operation apparatus 120 is installed externally, and the object information acquisition apparatus 100 is implemented by hardware separate from the operation apparatus 120, these apparatuses may be integrated into a single apparatus.

### Method of Defining Specified Region

A method of defining a specified region according to the present embodiment is described below. In the present embodiment, a signal of an acoustic wave generated inside a living body is also acquirable, and thus a characteristic distribution may be acquired not only in the form of a two-dimensional image (tomographic view) but also in the form of a three-dimensional image. FIG. 2A is a conceptual diagram illustrating a method of defining a specified region on a display screen in a process of acquiring a three-dimensional image. FIG. 2B is a conceptual diagram illustrating a three-dimensional region corresponding to the specified region in a living body.

As shown in FIG. 2A, a user may define a two-dimensional rectangle on the display screen on which a captured image is displayed thereby defining a specified region 201 to be scanned to acquire characteristic information. In the present embodiment, a mouse is used as the region specifying unit 123. A user is allowed to operate a cursor 204 to input a rectangle thereby defining a region with an arbitrary size. The user is allowed to view the captured image of the living body 101 during the operation of defining the specified region 201 on the display screen. This makes it easy for the user to correctly input the specified region 201 as is intended.

In FIG. 2B, a scanning region 303 is a region expressed in a scanning coordinate system of the receiver 104 and corresponding to the specified region 201 in a display coordinate system. In FIG. 2B, reference numeral 101 denotes a living body. A conceptual diagram on the right-hand side of FIG. 2B is a view seen in a direction denoted by an arrow shown in a diagram on the left-hand side of FIG. 2B. A three-dimensional region 306 is a region in which characteristic information is acquirable by scanning the receiver over the scanning region 303. The three-dimensional region 306 is determined by a distance between the holding units 102. During a measurement operation, the distance between the holding units 102 is maintained at a fixed value. When the specified region 201 is defined in the display coordinate system on the display screen as described above, the three-dimensional region 306 is determined as a three-dimensional specified region in an actual living body. That is, defining the specified region 201 in the display coordinate system results in defining the three-dimensional specified region in the three-dimensional coordinate system.

### Flow of Display Control Method

Next, referring to FIG. 3, a display control process according to the present embodiment is described below. FIG. 3 is a flow chart of the display control process according to the present embodiment.

After the apparatus is started, the process shown in the flow chart of FIG. 3 starts from a state in which a user inputs a specified region on the display screen on which an image of the living body 101 captured in a particular direction is displayed. That is, the display control method according to the present embodiment includes a step of first displaying a captured image of an object. In the present embodiment, a mouse is used as the region specifying unit 123.

In step S301, if a user presses down a mouse button at a particular point on the display screen, then this point is input as a start point of the specified region. In step S302, if the user changes the size of the specified region by dragging the mouse, then the changed size of the specified region is sent to the region calculation unit 109.

In step S303, the region calculation unit 109 converts the received expression of the specified region from the display coordinate system into the scanning coordinate system of the receiver. In step S304, the region correction unit 110 corrects the length of the specified region in the sub scanning direction expressed in the scanning coordinate system so as to be equal to an integral multiple of the sub-scanning width of the receiver. Note that in the present and other embodiments of the present invention, integral numbers are limited to positive ones. Thereafter, in step S305, the expression of the corrected specified region is converted from the scanning coordinate system into the display coordinate system and is transmitted to the display unit 124. The concepts of the process in steps S303 to S305 are described in further detail below.

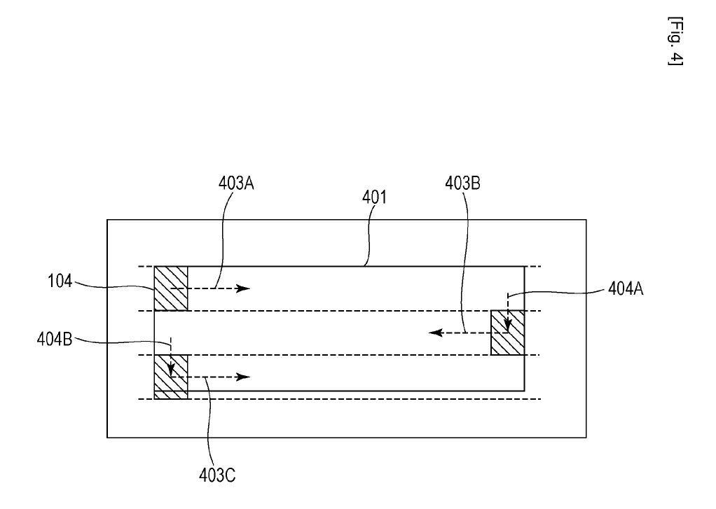

FIG. 4 is a conceptual diagram illustrating a specified region in a scanning coordinate system. In FIG. 4, it is assumed by way of example that the scanning width of the receiver 104 in the sub scanning direction is equal to the size of the receiver 104 in the sub scanning direction. It is also assumed that the size of the receiver 104 is equal to the size of an effective element array region in which a plurality of elements are disposed (a region in which elements are disposed for outputting reception signals used in generating image data). For example, in a case where the size of the receiver 104 in the sub scanning direction is equal to 5 cm, if main scanning is performed over one stripe, then characteristic information is obtained for the stripe with a width of 5 cm as measured in the sub scanning direction. Note that the width of sub scanning (404A and 404B in the example shown in FIG. 4) of the receiver is also 5 cm which is equal to the size of the receiver 104 in the sub scanning direction. Hereinafter, each region that is scanned in the main scanning direction while receiving an acoustic wave is referred to as a stripe. The sub-scanning width may be determined in advance or information associated with the scanning width may be given as required from the scanning-width information generation unit 131.

In the present embodiment, in a case where the length of the specified region 401 in the sub scanning direction expressed in the scanning coordinate system is, for example, 13 cm, the receiver 104 needs to perform main scanning for three stripes (that is, it is necessary to perform sub scanning two times) to acquire characteristic information of the specified region 401 with the above-described size. The description is continued below with a further assumption that the necessary length of the main scanning (403A, 403B, and 403C) is equal to the length of the specified region 401 in the scanning coordinate system.

On receiving the information associated with the specified region, the region correction unit 110 performs a correction such that the length of the specified region in the sub scanning direction in the scanning coordinate system is equal to 15 cm or 10 cm, i.e., such that the length of the specified region is corrected to the length of a first scanning region acquirable via main scanning for three stripes (via two sub scans) or the length of a second scanning region acquirable via main scanning for two stripes (via one sub scan).

Setting may be performed in advance in terms of which value is employed in the correction, or a condition set by the condition setting unit 132 may be given as required in response to a command issued by a user via a menu screen. More specifically, for example, in a case in which the length Z of the specified region in the sub scanning direction expressed in the scanning coordinate system is equal to or greater than 10 cm and less than 12.5 cm, the length may be corrected to 10 cm, while when Z is greater than 12.5 cm and equal to or less than 15 cm, the length may be corrected to 15 cm, i.e., the condition may be set with reference to a size one-half the scanning width. Alternatively, the correction may be performed such that when Z is greater than 10 cm and less than 15 cm, the length is corrected to 10 cm. In this case, as a matter of course, the length may be corrected to 15 cm instead of 10 cm.

That is, the process may include a step of setting whether the length of the specified region is corrected to the length of the first scanning region (that is to be scanned to acquire characteristic information of the specified region) or to the length of the second scanning region (in which the number of sub-scans is smaller by one than the number of sub-scans in the first scanning region). In the present embodiment, the scanning width of the receiver 104 in the sub scanning direction is equal to the size of the receiver 104. Therefore, in a case where the sub-scanning width of the receiver is L and the length Z of the specified region in the sub scanning direction satisfies nL<z<(n+1) L, the correction condition is set such that the length of the specified region in the sub scanning direction is adjusted to be equal to nL or (n+1) L, where n is a positive integer.

In the following description, it is assumed, by way of example, that the length of the specified region in the sub scanning direction in the scanning coordinate system is corrected to 15 cm, i.e., the length of the region acquirable via three main scans (two sub-scans).

In step S305, the region correction unit 110 transmits to the display unit 124 information associated with the corrected region converted from the scanning coordinate system into the display coordinate system. That is, information for displaying the adjusted region on the display unit 124 is output.

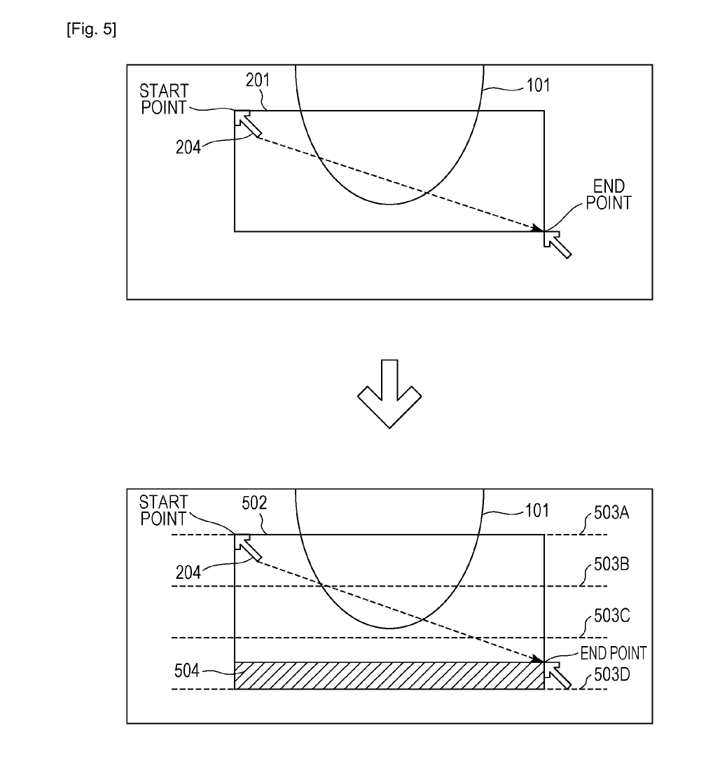

In step S306, the corrected region, i.e., the adjusted region is displayed on the display unit 124. FIG. 5 is a schematic diagram illustrating an example of an adjusted region displayed on the display unit 124. In this figure, a region 502 represents a corrected region acquirable via three main scans (two sub scans).

Lines 503A, 503B, 503C, and 503D (they will be generically referred to as lines 503 when it is not necessary to distinguish among them) are for indicating sub-scanning widths converted in expression into the display coordinate system from the scanning coordinate system. A region 504 represents a region added, in the correction by the region correction unit 110, to the specified region 201, i.e., a difference between the corrected region 502 (the adjusted region) and the specified region 201. Although this difference region is shown in FIG. 5 for convenience of illustration, this region may be actually displayed on the display screen in such a manner that a user can distinguish it from other regions, or this region may not be actually displayed.

Lines 503 indicating a sub scanning width may be displayed when the specified region 201 is defined using the region specifying unit 123. That is, in a state (a mode) in which a user is allowed to define the specified region, the display control unit 130 may display guide information including lines indicating the scanning width. That is, the display control unit 130 may output display information for displaying a guide indicating the scanning width. This makes it possible for a user to, during the operation of defining the specified region 201, recognize the sub-scanning width of the receiver which has a particular relationship with the number of times scanning is performed. In addition to or instead of information such as lines or the like indicating the scanning width, information directly indicating the number of scans may be displayed.

During the process of defining the specified region 201, i.e., in the state (mode) in which a user is allowed to define the specified region, not only the rectangle indicating the corrected region but also a rectangle indicating the size of the original specified region 201 may be displayed. That is, the display control unit 130 may output display information for displaying the original specified region. Displaying the size of the original specified region 201 makes it possible for a user to recognize the relationship between the original specified region 201 and the corrected region 502 during the process of defining the specified region 201.

By continuously performing steps S302 to S306, it is possible to display the corrected region such that a change in the specified region defined by the region specifying unit 123 is immediately reflected in the corrected region, and thus a user is allowed to recognize the scanning region of the receiver in real time.

In step S307, an end point of the specified region is input by releasing the mouse button. In the above example, in the defining the rectangle, the start point defines the upper-left corner of the rectangle and the end point defines the lower-right corner of the rectangle. Alternatively, the start point may define the upper-right corner and the end point may define the lower-left corner of the rectangle. When the end point is specified, the corrected region displayed is set as an acquisition region in which characteristic information is to be actually acquired by scanning the receiver.

In the example shown in FIG. 5, if a user drags the mouse, an image of a rectangle indicating the specified region 201 is displayed together with the captured image of the living body 101 in a superimposed manner. In a case where the specified region 201 is smaller than three times the sub-scanning width, the corrected region 502 is temporarily displayed in the form of an image of a rectangle in such a manner that it is superimposed on the captured image of the living body 101. It is allowed to change the size of the corrected region 502 at any time unless the mouse button is released. If the user releases the mouse button, the size of the image of the rectangle indicating the corrected region 502 is fixed and the resultant image is displayed.

The corrected region is adjusted so as to be equal to the length of the first or second scanning region in the sub scanning direction (more specifically, 10 cm or 15 cm, i.e., an integral multiple of the sub-scanning width 5 cm), and thus the length of the corrected region in the sub scanning direction becomes equal to the length of the actual scanning region of the receiver in the sub scanning direction. Thus, upon receiving the information associated with the corrected region transmitted from the display control unit 130 to the scan control unit 107, the scan control unit 107 scans the receiver in accordance with the received information associated with the corrected region. Thus, the object information acquisition apparatus 100 is allowed to acquire characteristic information of a three-dimensional region in the object corresponding to the corrected region.

Furthermore, in the present embodiment, instead of producing the adjusted region by correcting the length of the specified region, an adjusted region may be newly produced based on information associated with the length of the specified region (such that, for example, the length of the adjusted region may be equal to an integral multiple of a scanning width). That is, the correction is not limited to that performed by the display control unit 131. It is sufficient for the display control unit 131 to be capable of producing information for displaying the adjusted region on the display unit. In a case where the specified region defined by a user is equal to an integral multiple of the scanning width of the receiver, the information associated with the specified region may be directly output as information associated with the adjusted region.

### First Modified Example

In the present embodiment, the display control process is not limited to that described above. In the example described above, in step S303, the region calculation unit 109 converts the expression of specified region 201 from the display coordinate system into the scanning coordinate system, and then the region correction unit 110 corrects the specified region expressed in the scanning coordinate system and outputs the resultant corrected region. Alternatively, after the region calculation unit 109 converts the specified region into the expression in the display coordinate system, the region calculation unit 109 may calculate a first scanning region that needs to be scanned to acquire characteristic information of the specified region and may transmit the resultant first scanning region to the region correction unit 110. Upon receiving the information associated with the scanning region, the region correction unit 110 may adjust the scanning region according to a predetermined condition and may convert the scanning region into an expression in the display coordinate system.

This process is described in further detail below with reference to a specific example (first modified example). In this specific example, it is assumed, as in the process described above with reference to the flow chart, that the scanning width of the receiver 104 in the sub scanning direction is set to 5 cm, which is equal to the size of the receiver 104 in the sub scanning direction, and the main scanning length is equal to the length of the specified region in the scanning coordinate system.

In a case where the length of the specified region in the sub scanning direction expressed in the scanning coordinate system is greater than 10 cm and equal to or less than 15 cm, the receiver 104 needs to perform main scanning for three stripes (that is, it is necessary to perform sub scanning two times) to acquire characteristic information of the region with the above-described size. Therefore, the region calculation unit 109 performs a calculation to determine a first scanning region by correcting the length of the specified region in the sub scanning direction to 15 cm (i.e., a region coverable by main scanning of three stripes (two sub scans)).

In the present example, the correction condition of the region correction unit 110 is set with reference to 2.5 cm which is a size one-half the scanning width. In this case, the region correction unit 110 performs a correction such that when the length of the specified region in the sub scanning direction in the scanning coordinate system is greater than 12.5 cm, the first scanning region determined by the region calculation unit 109 is directly converted from the scanning coordinate system into the display coordinate system. The result is employed as the adjusted region and display information of the adjusted region is transmitted to the display unit 124. Conversely, when the length of the specified region in the sub scanning direction in the scanning coordinate system is equal to or greater than 12.0 cm and less than 12.5 cm, the region correction unit 110 corrects the scanning region such that a second scanning region coverable by main scanning of two stripes (one sub-scan) is employed as the scanning region. The resultant corrected scanning region is converted from the scanning coordinate system into the display coordinate system, and information associated with the corrected region is transmitted, as display information of the adjusted region, to the display unit 124.

In the present modified example, as described above, after the first scanning region is determined by the calculation based on the specified region, the first scanning region is directly employed as the adjusted region or the first scanning region is corrected to the second region and output as the adjusted region depending on the correction condition. The criterion for the correction condition is not limited to the size one-half the scanning width. As in the control process described above with reference to the flow chart, the correction condition may be determined in advance, or a correction condition may be set by the condition setting unit 132 as required according to a command input by a user.

In the previous example and also in the present modified example, the display control process is performed such that the specified region is converted from the display coordinate system into the scanning coordinate system and corrected, and the corrected region is converted back from the scanning coordinate system into the display coordinate system. Alternatively, the specified region may be directly corrected while maintaining it in the display coordinate system. In this case, it is necessary to convert the scanning width from the scanning coordinate system into the display coordinate system.

According to the present embodiment and modified example, as described above, a user is allowed to intuitively recognize the necessary scanning region of the receiver corresponding to the specified region defined by the user. This makes it possible to reduce the possibility that a slight difference in the specified region results in an unnecessary increase in the number times the receiver is scanned, which may create a redundant time period in the acoustic wave reception time period for acquiring characteristic information, which may in turn redundantly increase a time period during which a person under examination is constrained.

### Second Modified Example

A second modified example of the first embodiment is described below. In this modified example, an explanation is given for a case where the scanning width of the receiver in the sub scanning direction is greater than the length of the receiver in the sub scanning direction, and a scanning path of the receiver in the main scanning direction overlaps a scanning path in the sub scanning direction. In the present modified example, the object information acquisition system is configured in a similar manner to that shown in FIG. 1, and thus a further description thereof is omitted. The processing flow of the display control is basically similar to that shown in FIG. 3, although there is some difference in the process in step S304 and following S304 as described below.

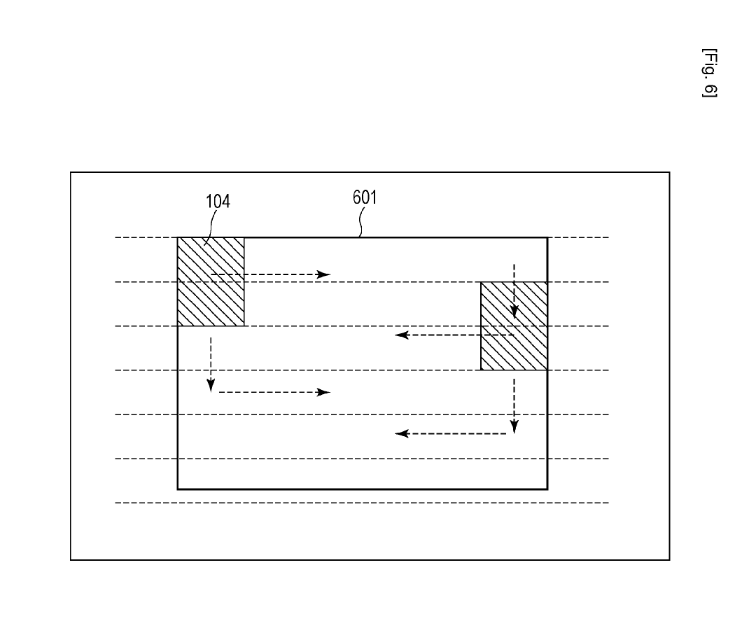

FIG. 6 is a conceptual diagram illustrating a specified region in a scanning coordinate system. In the example shown in FIG. 6, it is assumed, by way of example, that the scanning width of the receiver 104 in the sub scanning direction is 2.5 cm, and the length of the receiver 104 in the sub scanning direction is 5 cm. In this example, it is further assumed that the size of the receiver 104 is equal to the size of the effective element array region in which a plurality of elements are disposed.

In a case where the length of the specified region 601 expressed in the scanning coordinate system in the sub scanning direction is, for example, 13 cm, the receiver 104 needs to perform main scanning for five stripes (that is, it is necessary to perform sub scanning four times) to acquire characteristic information of the region with the above-described size. In the following description, it is assumed that the necessary length in the main scanning direction is equal to the length of the specified region 401 in the scanning coordinate system.

Thus, the region correction unit 110 performs a correction such that the length of the specified region in the sub scanning direction in the scanning coordinate system is equal to 15 cm or 12.5 cm, i.e., such that the length is equal to the length of a first scanning region acquirable via main scanning of five stripes (via four sub scans) or to the length of a second scanning region acquirable via main scanning for four stripes (via three sub scans). Setting may be performed in advance in terms of which value is employed in the correction, or a correction condition may be set by the condition setting unit 132 as required according to a command input by a user.

In the present modified example, as described above, the sub-scanning width of the receiver is smaller than the length of the receiver in the sub scanning direction. This is for making it possible for the signal processing unit to cumulatively add a plurality of reception signals to improve SNR. More specifically, when two or more different elements of the plurality of elements of the receiver receive acoustic waves from the object at the same scanning position at different points of time, electric signals output from the respective two or more different elements are cumulatively added together. The scanning width of the receiver is determined by the number of cumulative addition operations for the plurality of reception signals (the number of times the cumulative addition operation is performed). The smaller the scanning width, the greater the overlapping of the scanning region. Therefore, the smaller the scanning width, the greater the number of cumulative addition operations and thus SNR is more improved. Alternatively, to improve SNR, characteristic information may be cumulatively combined instead of cumulatively adding reception signals. More specifically, each time sub scanning is performed, the image forming unit acquires characteristic information of one stripe using a plurality of electric signals output from a plurality of elements. The image forming unit combines together characteristic information of a plurality of stripes to acquire a characteristic distribution in the object. That is, the image forming unit combines (by means of addition, multiplication, etc.) characteristic information acquired at the same position in the object. The number of times characteristic information is combined determines the scanning width of the receiver. Note that it may be allowed to perform both the cumulative addition of reception signals and the cumulative combination of characteristic information.

Thus, when the scanning-width information generation unit 131 receives a command from a user in terms of the number of cumulative addition operations or the number of cumulative combination operations, the scanning-width information generation unit 131 generates information associated with the scanning width of the main scanning and that of the sub scanning. The generated information is transmitted to the region correction unit 110.

The scanning trajectory described above with reference to FIG. 6 includes only one forward scan per one stripe. Alternatively, one stripe may be scanned in both forward and backward directions, or may be scanned a plurality of times. A stripe on the top and a stripe on the bottom are subjected to a less number of cumulative addition operations than the other stripes, and thus the number of scans may be increased for the stripe on the top and that on the bottom.

In FIG. 4 and FIG. 6, it is assumed that the size of the receiver 104 is equal to the size of the effective element array region in which a plurality of elements are disposed. However, the effective element array region may be smaller than the size of the receiver 104 (as in a case where dummy elements that do not use reception signals are disposed in a peripheral area). In such a case, the scanning width may be calculated based on the size of the effective element array region and the specified region may be corrected. In this case, a region obtained by correcting the specified region based on the scanning width calculated based on the size of the effective element array region may be displayed as the adjusted region.

### Second Embodiment

A second embodiment of the present invention is described below. In this embodiment, an adjusted region is obtained by adjusting the length of the specified region in the main scanning direction so as to correspond to an integral multiple of the main scanning width, and the resultant adjusted region is displayed on the display unit. That is, in the present embodiment, the first direction is taken in the main scanning direction and the second direction is taken in the sub scanning direction.

In the present embodiment, the object information acquisition system is configured in a similar manner to that according to the first embodiment, although there is a difference in function of the display control unit 130. Thus, a further description of similar parts to those in the first embodiment is omitted, and the following description focuses on differences from the first embodiment. The processing flow of the display control is similar to that described above with reference to FIG. 3, although there is some difference in the process in step S303 and following S303 as described below.

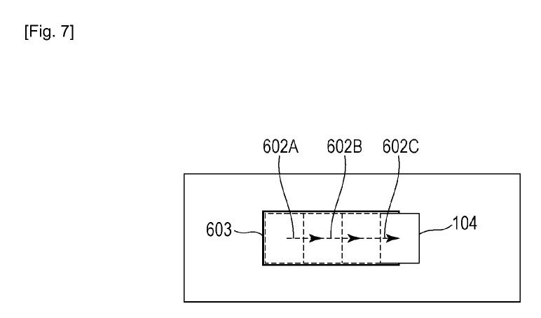

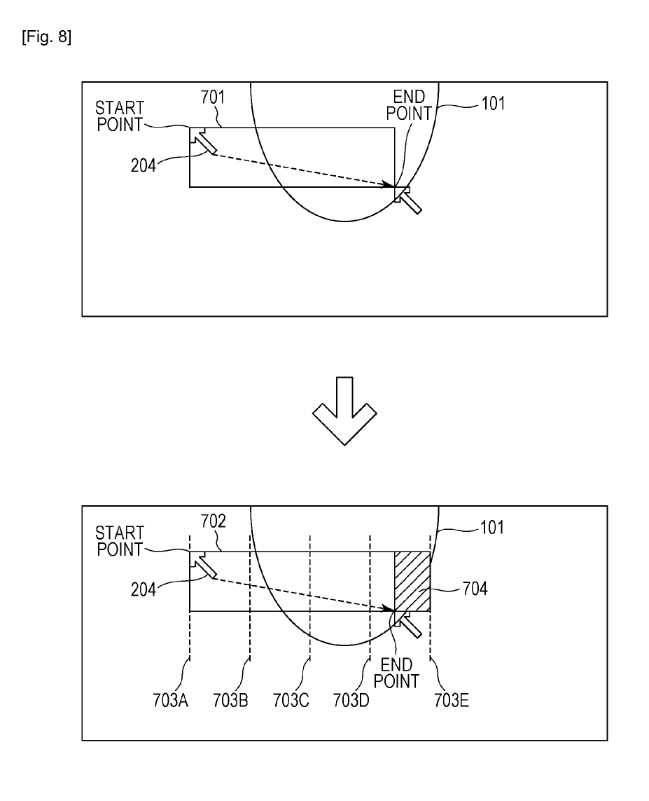

First, in the region calculation unit 109, a specified region 701 (see FIG. 8) defined on a captured image by the region specifying unit 123 is converted from the display coordinate system into the scanning coordinate system (step S303). FIG. 7 is a conceptual diagram illustrating a specified region in the scanning coordinate system. The number of times the main scanning of the receiver is performed is determined from the specified region in the scanning coordinate system.

In the example shown in FIG. 7, the scanning width of the receiver 104 in the main scanning direction is equal to the size of the receiver 104 in the main scanning direction. The receiver 104 receives an acoustic wave at each scanning position. Note that the size of the receiver 104 is equal to the size of the effective element array region. For example, when the size of the receiver 104 in the main scanning direction is 5 cm, the scanning width (602A, 602B, and 602C in FIG. 7) of the receiver in the main scanning direction is also equal to 5 cm. The scanning width may be determined in advance or may be determined as required according to information on the scanning width generated by the scanning-width information generation unit 131.

In a case where the length of the specified region 603 in the main scanning direction expressed in the scanning coordinate system is, for example, 18 cm, to acquire characteristic information of the region with this size, acoustic waves are received while shifting the scanning position of the receiver in the main scanning direction. More specifically, the main scanning of the receiver is performed three times, and thus acoustic waves are received at four different scanning positions. In this case, sub scanning is not necessary.

Next, in the region correction unit 110, the length of the specified region in the main scanning direction expressed in the scanning coordinate system is corrected so as to correspond to an integral multiple of the main scanning width of the receiver (step S304). More specifically, the length of the specified region in the main scanning direction in the scanning coordinate system is corrected to 20 cm or 15 cm, i.e., to the length of the first scanning region coverable at four scanning positions (via three main scans) or to the length of the second scanning region coverable at three scanning positions (via two main scans).

Setting may be performed in advance as to which value is to be employed in the correction, or a correction condition may be set by the condition setting unit 132 as required according to a command input by a user. The correction condition may be based on the value one-half the scanning width. More specifically, for example, when the length Z of the specified region in the main scanning direction is equal to or greater than 15 cm and less than 17.5 cm, the length may be corrected to 15 cm, while when Z is greater than 17.5 cm and equal to or less than 20 cm, the length may be corrected to 20 cm. Alternatively, for example, when Z is greater than 15 cm and less than 20 cm, the length may always be corrected 15 cm or may always be corrected to 20 cm.

That is, as in the first embodiment, a processing step may be provided to correct the length of the specified region to the length of the first scanning region or the length of the second scanning region. In the following description, it is assumed, by way of example, that the length of the specified region in the main scanning direction in the scanning coordinate system is corrected to 20 cm, i.e., the length of a region coverable at four scanning positions (via three main scans).

In step S305, the region correction unit 110 transmits, to the display unit 124, display information for displaying an adjusted region, i.e., a corrected region converted from the scanning coordinate system into the display coordinate system.

In step S306, the corrected region is displayed on the display unit 124. FIG. 8 is a schematic diagram illustrating a specified region 701 and a corrected region 702. In this example shown in FIG. 8, the corrected region 702 indicates a region acquirable via three main scans (two sub scans).

Lines 703A, 703B, 703C, 703D, and 703E (the term a “line 703” or “lines 703” will be used when distinguishment among them is not necessary) are for indicating main scanning width of the receiver converted from the scanning coordinate system into the display coordinate system. A region 704 represents a region added to the specified region 701 as a result of the correction by the region correction unit 110, i.e., a difference between the corrected region 702 (the adjusted region) and the specified region 701. Although this difference region is shown in FIG. 8 for convenience of illustration, this region may be actually displayed on the display screen in such a manner that a user can distinguish it from other regions, or this region may not be actually displayed.

The lines 703 indicating the main scanning width may be displayed when the specified region 701 is defined using the region specifying unit 123. This makes it possible for a user to recognize the main scanning width of the receiver during the operation of defining the specified region 701. In addition to or instead of information such as lines or the like indicating the scanning width, information directly indicating the number of scans may be displayed. During the process of defining the specified region 701, a rectangle indicating the size of the original specified region 701 may also be displayed. Displaying the rectangle indicating the size of the original specified region 701 makes it possible for a user to recognize the relationship between the specified region and the corrected region during the process of defining the specified region 701.

By continuously performing steps S302 to S306, it is possible to display the corrected region such that a change in the specified region defined by the region specifying unit 123 is immediately reflected in the corrected region, and thus a user is allowed to recognize the scanning region of the receiver in real time.

In step S307, an end point of the specified region is input by releasing the mouse button. When the end point is specified, the corrected region displayed is set as an acquisition region in which characteristic information is to be actually acquired by scanning the receiver. Information associated with the corrected region is transmitted from the display control unit 130 to the scan control unit 107, and the scan control unit 107 scans the receiver based on the corrected region. The scanning of the receiver may be performed in a step-and-repeat manner in which the receiver stops at each scanning position, receives an acoustic wave, and then moves to a next scanning position. Note that, in the main scanning direction, the receiver may be continuously moved at a constant speed. In a case where the receiver is continuously scanned, each scanning position is defined as a position where the receiver is located when light is irradiated. The number of strokes of main scanning is defined by the number of times the receiver moves from one scanning position to a next scanning position in one stripe.

In the above-described manner, the object information acquisition apparatus 100 is capable of acquiring three-dimensional characteristic information in the object corresponding to the corrected region.

As described above, the length of the specified region in the main scanning direction defined by a user is corrected a user is allowed to intuitively recognize the necessary scanning region of the receiver corresponding to the specified region. In the present embodiment, as in the first embodiment, instead of producing the adjusted region by correcting the length of the specified region, an adjusted region may be newly produced based on information associated with the length of the specified region (such that, for example, the length of the adjusted region may be equal to an integral multiple of a scanning width). That is, the correction is not limited to that performed by the display control unit 131. It is sufficient for the display control unit 131 to be capable of producing information for displaying the adjusted region on the display unit. In a case where the specified region defined by a user is equal to an integral multiple of the scanning width of the receiver, the information associated with the specified region may be directly output as information associated with the adjusted region.

In the present embodiment, as in the first modified example of the first embodiment, after the region calculation unit 109 converts the specified region into the expression in the display coordinate system, the region calculation unit 109 may calculate a first scanning region necessary to acquire the specified region and may transmit the resultant first scanning region to the region correction unit 110. Upon receiving the information associated with the first scanning region, the region correction unit 110 may generate a corrected region based on a determined condition and may convert the scanning region into an expression in the display coordinate system.

Alternatively, the specified region may be directly corrected while maintaining it in the display coordinate system. In this case, it is necessary to convert the scanning width from the scanning coordinate system into the display coordinate system.

Also in the present embodiment, as in the second modified example of the first embodiment, the receiver may be scanned in the main scanning direction at overlapping scanning positions and a plurality of reception signals may be cumulatively added together to improve SNR. That is, the present embodiment may also be applied to a case in which the scanning width of the receiver in the main scanning direction is smaller than the length of the receiver in the main scanning direction. In the display control processing, as in the second modified example of the first embodiment, the scanning-width information generation unit 131 generates information associated with the scanning width of the main scanning in accordance with a command issued by a user in terms of the number of times reception signals are cumulatively added, the number of times characteristic information (image data) is cumulatively combined, etc., and the scanning-width information generation unit 131 transmits the generated information to the region correction unit 110. By overlapping scanning positions in the main scanning direction in the above-described manner, it becomes possible for the object information acquisition apparatus 100 to acquire image data with improved SNR.

### Third Embodiment

In a third embodiment described below, a specified region is adjusted in terms of length in both the main scanning direction and the sub scanning direction, and a result is displayed as an adjusted region. In the present embodiment, to correct the length of the specified region in both the main scanning direction and the sub scanning direction, the function of the region calculation unit 109 and the function of region correction unit 110 according to the first embodiment and those according to the second embodiment are combined.

The region calculation unit 109 calculates the number of sub-scans based on information associate with the specified region as in the process according to the first embodiment, and calculates the number of main scans as in the process according to the second embodiment.

The region correction unit 110 corrects the length of the specified region in the sub scanning direction based on the sub-scanning width as in the process according to the first embodiment, and then the region correction unit 110 corrects the length of the specified region in the main scanning direction as in the process according to the second embodiment. Thereafter, the region correction unit 110 outputs information for displaying the corrected region on the display unit 124.

By displaying, on the display unit, the adjusted region obtained by adjusting the length of the specified region defined by an use in both the main scanning direction and the sub scanning direction, it becomes possible for the user to intuitively recognize the necessary scanning region of the receiver corresponding to the specified region. This makes it possible to reduce the possibility that a slight difference in the specified region results in an unnecessary increase in the number of strokes of scanning of the receiver, which may create a redundant time period in the acoustic wave reception time period for acquiring characteristic information, which may in turn redundantly increase a time period during which a person under examination is constrained.

### Fourth Embodiment

In the first to third embodiments described above, the object information acquisition apparatuses and systems are disclosed which are based on the photoacoustic effect, that is, which acquire object information by illuminating object with light and receiving an acoustic wave generated in the object. Alternatively, an object information acquisition apparatus or a system may be realized using an ultrasonic wave echo. In this case, an ultrasonic wave is transmitted toward an object, and the ultrasonic wave reflected back from the inside of the object is received as an acoustic wave by a receiver. In the case of an apparatus using an ultrasonic wave echo, the receiver may be configured such that it also functions as a transmitter that transmits an ultrasonic wave to an object.

### Fifth Embodiment

The present invention may be practiced by executing the following processing. That is, software (program) for implementing the functions disclosed in the embodiments is provided to a system or an apparatus via a network or a storage medium, and a computer (or a CPU or an MPU) in the system or the apparatus reads and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-172974, filed Aug. 8, 2011 and Application No. 2012-156629, filed Jul. 12, 2012, which are hereby incorporated by reference herein in their entirety.

### REFERENCE SIGNS LIST

100 Object information acquisition apparatus
101 Living body
102 Holding unit
103 Illumination unit
104 Receiver
105 Measurement unit
106 Signal processing unit
107 Scan control unit
108 Camera
109 Region calculation unit
110 Region correction unit
120 Operation apparatus
121 Image forming unit
122 Display unit
123 Region specifying unit
124 Display unit
130 Display control unit
131 Scanning-width information generation unit
132 Condition setting unit

1. An object information acquisition apparatus configured to receive an acoustic wave from an object and acquire characteristic information of the object, comprising:
a receiver configured to receive the acoustic wave and convert the received acoustic wave into an electric signal;
a scan control unit configured to scan the receiver at least in one direction; and a display control unit,
wherein the display control unit receives information associated with a specified region defined by a user as a region in which characteristic information is to be acquired, and the display control unit outputs information for displaying an adjusted region obtained by adjusting the specified region such that a length of the specified region in a first direction corresponds to an integral multiple of a scanning width of the receiver in the first direction.

2. The object information acquisition apparatus according to claim 1, wherein in a case where the length of the specified region in the first direction is greater than nL and smaller than (n+1)L where L is the scanning width in the first direction and n is a positive integer, the display control unit outputs information for displaying, on the display unit, the adjusted region obtained by adjusting the specified region such that the length of the specified region in the first direction corresponds to nL or (n+1)L.

3. The object information acquisition apparatus according to claim 1, wherein the scan control unit scans the receiver in two directions,
and wherein the first direction is a sub scanning direction crossing a main scanning direction in which the receiver is scanned while receiving an acoustic wave at each reception position.

4. The object information acquisition apparatus according to claim 1, wherein:
the receiver includes a plurality of elements each configured to receive the acoustic wave and convert the received acoustic wave into an electric signal; and
the display control unit determines a scanning width of the receiver in the first direction based on the number of times electric signals output from the respective two or more different elements are cumulatively added when the two or more different elements of the plurality of elements receive acoustic waves from the object at the same scanning position at different points of time.

5. The object information acquisition apparatus according to claim 3, wherein
the receiver includes a plurality of elements each configured to receive the acoustic wave and convert the received acoustic wave into an electric signal, and wherein
the object information acquisition apparatus further comprises a characteristic information acquisition unit configured to acquire characteristic information of one stripe using the plurality of electric signals output from the plurality of elements each time the sub scanning is performed and combine characteristic information of a plurality of stripes thereby acquiring a characteristic distribution in the object, and wherein
the display control unit determines the scanning width of the receiver in the first direction based on the number of times characteristic information at the same position in the object in the characteristic distribution of each stripe is combined.

6. The object information acquisition apparatus according to claim 1, wherein in a state in which it is allowed to define the specified region, the display control unit further displays, on the display unit, information indicating the scanning width of the receiver in the first direction or the number of times the receiver is scanned in the first direction.

7. The object information acquisition apparatus according to claim 1, wherein in a state in which it is allowed to define the specified region, the display control unit further displays the specified region on the display unit.

8. The object information acquisition apparatus according to claim 1, further comprising an image capturing unit configured to capture an image of the object, wherein
in a state in which it is allowed to define the specified region, the display control unit further displays the captured image of the object on the display unit.

9. The object information acquisition apparatus according to claim 1, wherein
the receiver includes a plurality of elements each configured to receive the acoustic wave and convert the received acoustic wave into an electric signal, wherein
the object information acquisition apparatus further comprises a characteristic information acquisition unit configured to acquire a characteristic information in the object using a plurality of electric signals output from the plurality of elements, wherein
the scan control unit scans the receiver based on information output from the display control unit, and wherein
the characteristic information acquisition unit acquires characteristic information of a three-dimensional region in the object using a plurality of electric signals obtained via scanning of the receiver.

10. An object information acquisition apparatus configured to receive an acoustic wave from an object and acquire characteristic information of the object, comprising:
a receiver configured to receive the acoustic wave and convert the received acoustic wave into an electric signal;
a scan control unit configured to scan the receiver at least in one direction; and
a display control unit,
wherein the display control unit receives information associated with a specified region defined by a user as a region in which characteristic information is to be acquired, and the display control unit outputs information for displaying, on the display unit, an adjusted region obtained by adjusting the length of the specified region in the first direction so as to correspond to a length of a first scanning region in the first direction in which a receiver needs to be scanned to acquire characteristic information of the specified region or a length of a second scanning region in the first direction in which the number of scans in the first direction is smaller by one than in the first scanning region.

11. The object information acquisition apparatus according to claim 1, further comprising a light source configured to emit light, wherein
the receiver receives an acoustic wave generated by illuminating the object with the light.

12. An object information acquisition system comprising:
the object information acquisition apparatus according to claim 1;
a display unit configured to display a captured image of the object; and
a region specifying unit for use by a user to define the specified region.

13-22. (canceled)

23. A method of displaying a region in which characteristic information of an object is to be acquired, comprising:
displaying a captured image of the object; and
displaying a specified region and an adjusted region, the specified region defined by a user as the region in which the characteristic information of the object is to be acquired, the adjusted region obtained by adjusting the specified region such that at least the length of the specified region in a first direction corresponds to an integral multiple of a scanning width, in the first direction, of a receiver configured to receive an acoustic wave from the object.

24. The method according to claim 23, wherein the adjusted region displayed is adjusted such that in a case where the length of the specified region in the first direction is greater than nL and smaller than (n+1)L where L is the scanning width in the first direction and n is a positive integer, the length of the specified region in the first direction corresponds to nL or (n+1)L.

25. The method according to claim 23, wherein the first direction is a sub scanning direction crossing a main scanning direction in which the receiver is scanned while receiving an acoustic wave at each reception position.

26. The method according to claim 23, further comprising displaying information indicating the scanning width of the receiver in the first direction or the number of times the receiver is scanned in the first direction.

27. A program configured to control a computer to execute processing steps according to claim 23.
